(19) United States
(12) Patent Application Publication
(10) Pub. No.: US 2021/0085899 A1
(43) Pub. Date: Mar. 25, 2021

(54) MEDICAL DEVICE FOR NEGATIVE PRESSURE VENTILATION

(71) Applicant: PHYSIO-CONTROL, INC., Redmond, WA (US)

(72) Inventors: Tyson Taylor, Bothell, WA (US); Benjamin Danziger, Kenmore, WA (US); Ryan Bowman, Richland, WA (US); Neal Clark, Snohomish, WA (US)

(73) Assignee: PHYSIO-CONTROL, INC., Redmond, WA (US)

(21) Appl. No.: 17/028,594

(22) Filed: Sep. 22, 2020

Related U.S. Application Data
(60) Provisional application No. 62/905,074, filed on Sep. 24, 2019.

(51) Int. Cl. A61M 16/00

(52) U.S. Cl. CPC A61M 16/0096; A61M 16/0009; A61M 2230/432; A61M 16/022; A61M 2205/502; A61M 16/0072

(57) ABSTRACT

An exemplary example of a medical device can include a retention structure for at least partially encircling a patient's body, the retention structure including a central member and a support portion configured to be placed underneath a patient, a piston extending from the central member, a driver coupled to the piston configured to retract and extend the piston, a patient contact member attached to the piston, the patient contact member configured to adhere to the patient's body, and a controller. The controller can be configured to cause the driver during a session to perform at least two cycles of negative pressure ventilation, each of the at least two cycles of negative pressure ventilation including positioning the piston at a reference position, retracting the piston from the reference position to an expansion position to expand a chest of a patient to generate negative pressure ventilation, and returning the piston from the expansion position to the reference position.

### PRIORITY

This disclosure claims benefit of U.S. Provisional Application No. 62/905,074, titled “MEDICAL DEVICE FOR NEGATIVE PRESSURE VENTILATION,” filed on Sep. 24, 2019, which is incorporated by reference in its entirety.

### FIELD

The present disclosure relates to a system and method of delivering negative pressure ventilation.

### BACKGROUND

Emergency airway management for patients experiencing respiratory distress is a relatively common but high-risk procedure. Even in preplanned surgical procedures requiring a managed airway, the risk of significant adverse events is small but not negligible. In the unplanned emergent setting the risk of adverse outcomes—including cardiac arrest and death—are significant. The risk for catastrophic outcomes is primarily driven by the need to place a device in the patient's airway which simultaneously allows ventilation, maintenance of airway patency, and protection from aspiration of bodily fluids such as vomit.

Although many approaches to managing an airway exist, the current gold standard is endotracheal intubation. In the emergent setting, a procedure termed rapid sequence intubation (RSI) is performed. RSI is a complex multistage process which typically includes the use of paralytics and sedatives and is arguably one of the most advanced medical procedures performed by paramedics.

The use of paralytics during RSI and the need to place a device in the airway guarantees a period of apnea during which no positive pressure ventilation is provided with oxygen saturation hopefully being maintained by a combination of preoxygenation and passive oxygen insufflation. Apnea occurs because the pre-intubation bag-valve-mask ventilation needs to be stopped to visualize and place the endotracheal tube and ventilation only resumes if the intubation process is either completed successfully or paused to maintain oxygen saturation.

Even with highly trained care providers following best practices there are uncertainties that arise during the RSI that can lead to oxygen desaturation and adverse outcomes. Such risks could be mitigated or even eliminated if a method or device for ventilating the patient, while the endotracheal tube or other advanced airway device is being placed, existed.

### SUMMARY

An exemplary example of a medical device can include a retention structure for at least partially encircling a patient's body, the retention structure including a central member and a support portion configured to be placed underneath a patient, a piston extending from the central member, a driver coupled to the piston configured to retract and extend the piston, a patient contact member attached to the piston, the patient contact member configured to adhere to the patient's body, and a controller. The controller can be configured to cause the driver during a session to perform at least two cycles of negative pressure ventilation, each of the at least two cycles of negative pressure ventilation including positioning the piston at a reference position, retracting the piston from the reference position to an expansion position to expand a chest of a patient to generate negative pressure ventilation, and returning the piston from the expansion position to the reference position.

In some examples, the controller can be further configured to perform at least two cycles of cardiopulmonary resuscitation (CPR), each of the at least two cycles of CPR including positioning the piston at a reference position, extending the piston from the reference position to a compression position to compress a chest of a patient, and returning the piston from the compression position to the reference position.

Additionally or alternatively, an exemplary example of a medical device can include a negative pressure ventilation mechanism configured to perform successive negative pressure ventilations on a chest of a patient, the negative pressure ventilation mechanism including a support portion configured to be placed underneath a patient, a piston, a patient contact member attached to the piston and configured to adhere to the patient's body, and a driver coupled to the piston configured to retract and extend the piston, and a controller communicatively coupled with the negative pressure ventilation mechanism. The controller can be configured to receive at least one input, determine whether at least one of a depth, a waveform, or a rate of negative pressure ventilation should be adjusted based on the at least one input, and responsive to a determination that at least one of the depth of negative pressure ventilation or the rate of negative pressure ventilation should be adjusted, cause the driver to adjust at least one of the depth, the waveform, or the rate of negative pressure ventilation.

Additionally or alternatively, an exemplary example of a medical device can include a negative pressure ventilation mechanism configured to perform successive negative pressure ventilations on a chest of a patient, the negative pressure ventilation mechanism including a support portion configured to be placed underneath a patient, a piston, a patient contact member attached to the piston and configured to adhere to the patient's body, and a driver coupled to the piston configured to retract and extend the piston; and a controller communicatively coupled with the negative pressure ventilation mechanism. The controller can be configured to cause the driver to retract and extend the piston at a depth, a waveform, and a rate, receive at least one input, determine whether a predetermined condition is met, responsive to a determination that the predetermined condition is met, cause a prompt to be issued for advising a rescuer to cause one or more of the depth, the waveform, or the rate to be adjusted.

Additionally or alternatively, an exemplary example of a medical device can include a retention structure for at least partially encircling a patient's body, the retention structure including a central member and a support portion configured to be placed underneath a patient, a piston extending from the central member, a patient contact member attached to the piston, the patient contact member configured to adhere to the patient's body, a sensor configured to detect the patient contact member, a driver coupled to the piston configured to retract and extend the piston, and a controller communicatively coupled with the driver. The controller can be configured to receive at least one input from the sensor, determine whether the patient contact member is configured for CPR or negative pressure ventilation based on the at least one input, and responsive to the determination, cause the driver to perform a CPR or negative pressure ventilation protocol.

Additionally or alternatively, an exemplary example of a medical device can include a retention structure for at least partially encircling a patient's body, the retention structure including a central member and a support portion configured to be placed underneath a patient, a piston extending from the central member, a driver coupled to the piston configured to retract and extend the piston, a patient contact member attached to the piston, the patient contact member configured to adhere to the patient's body, and a controller configured to cause the driver during a session to alternatively perform a cycle of negative pressure ventilation, a first cycle of CPR immediately following the first cycle of negative pressure ventilation, and a second cycle of negative pressure ventilation immediately following the first cycle of CPR. Each of the first and second cycles of negative pressure ventilation can include positioning the piston at a reference position, retracting the piston from the reference position to an expansion position to expand a chest of a patient to generate negative pressure ventilation, and returning the piston from the expansion position to the reference position. Each of the first and second cycles of CPR can include positioning the piston at the reference position, extending the piston from the reference position to a compression position to compress a chest of a patient, and returning the piston from the compression position to the reference position. Although a one to one ratio is discussed here, examples of the disclosure are not limited to such a ratio, as is described in more detail below. In some examples, one or more cycles of negative pressure ventilation may be performed, followed by one or more cycles of CPR.

Additionally or alternatively, an exemplary example of a medical device can include a piston having a slot for a ventilation bag extending from a central member, a driver coupled to the piston configured to retract and extend the piston, and a controller. The controller can be configured to cause the driver to extend the piston to compress a chest of a patient and retract the piston to a first position during a chest compression and to cause the drive to retract the piston to a second position during a ventilation to cause the ventilation bag in the slot to compress against the central member.

Additionally or alternatively, an exemplary example of a medical device can include a compression mechanism, a backboard, a containment device coupled to the backboard, the containment device structured to retain a ventilation bag, and a controller configured to instruct the compression mechanism to compress the ventilation bag during a ventilation mode.

In some examples, the containment device can include a first segment structured to attach to a first leg of the medical device and the backboard and a second segment structured to attach to a second leg of the medical device and the backboard. Additionally or alternatively, the containment device can be a removable rectangular containment device to allow the medical device to also operate as a chest compression device. The containment device can also include openings on two opposing sides of the rectangular containment device in some examples to accommodate various tubes, sensors, and/or valves connected to the ventilation bag.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a diagram of components of an abstracted medical device according to the present disclosure.

FIG. 2 is an exemplary medical device including a piston and a suction cup according to the present disclosure.

FIG. 3 is a flow chart of operation of an example of a medical device including etCO2 sensor data.

FIG. 4 is a flow chart of operation of an example of a medical device including SpO2 sensor data.

FIG. 5 is a flow chart of operation of an example of a medical device including ECG sensor data.

FIG. 6 is a flow chart of operation of an example of a medical device including SpO2 and etCO2 sensor data.

FIG. 7 is a flow chart of operation of an example of a medical device including SpO2 and ECG sensor data.

FIG. 8 is a flow chart of operation of an example of a medical device including SpO2, ECG and etCO2 sensor data.

FIG. 9 is a flow chart of operation of an example of a medical device including invasive blood pressure sensor data.

FIG. 10A is a side view of an exemplary contact surface.

FIG. 10B is a top view of the exemplary contact surface of FIG. 10A.

FIG. 11 is a top view of an exemplary contact surface.

FIG. 12 is a top view of an exemplary contact surface.

FIG. 13 is a side view of an exemplary contact surface

FIG. 14 is a table of exemplary initial tidal volume and frequency for negative pressure ventilation.

FIG. 15 is an exemplary medical device including a piston having a slot to accommodate a ventilation bag according to the present disclosure.

FIG. 16 is an exemplary medical device for compressing a ventilation bag during a ventilation mode.

FIG. 17 is an exemplary backboard to connect to the medical device of FIG. 2 to compress a ventilation bad during a ventilation mode.

### DETAILED DESCRIPTION

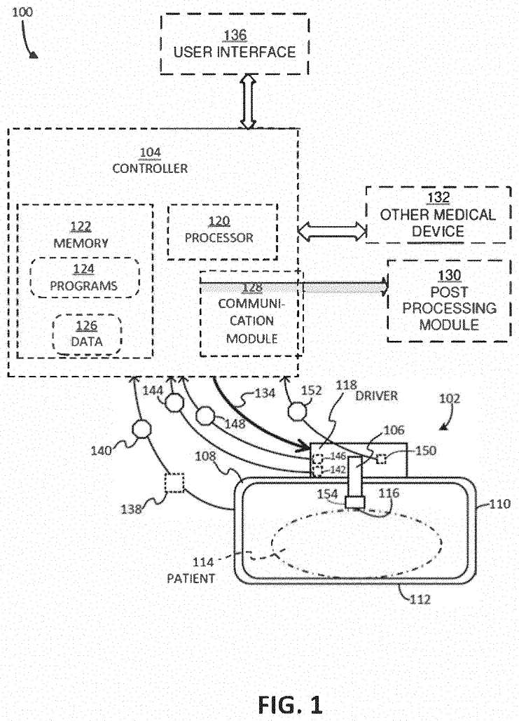

FIG. 1 illustrates an example schematic block diagram of a medical device 100. As will be understood by one skilled in the art, the medical device 100 may include additional components not shown in FIG. 1. The medical device 100 includes a controller 104, which may be in electrical communication with a negative pressure ventilation mechanism 102. Although called a negative pressure ventilation mechanism 102, the negative pressure ventilation mechanism 102 may also perform high frequency ventilations, using expansion or compression of a patient's chest, or may perform compressions on a ventilation bag, each of which will be discussed in more detail below. The negative pressure ventilation mechanism 102 can include at least one of a chest or abdomen manipulation element configured to compress and/or expand at least one of a patient's chest or abdomen, such as a piston based manipulation device. The manipulation element in FIG. 1 includes a piston 106 and a contact member 154. Contact member 154 can include a suction cup, a manipulation pad, or other device configured to make contact with a patient's chest. In some examples, contact member 154 is disposable after each use.

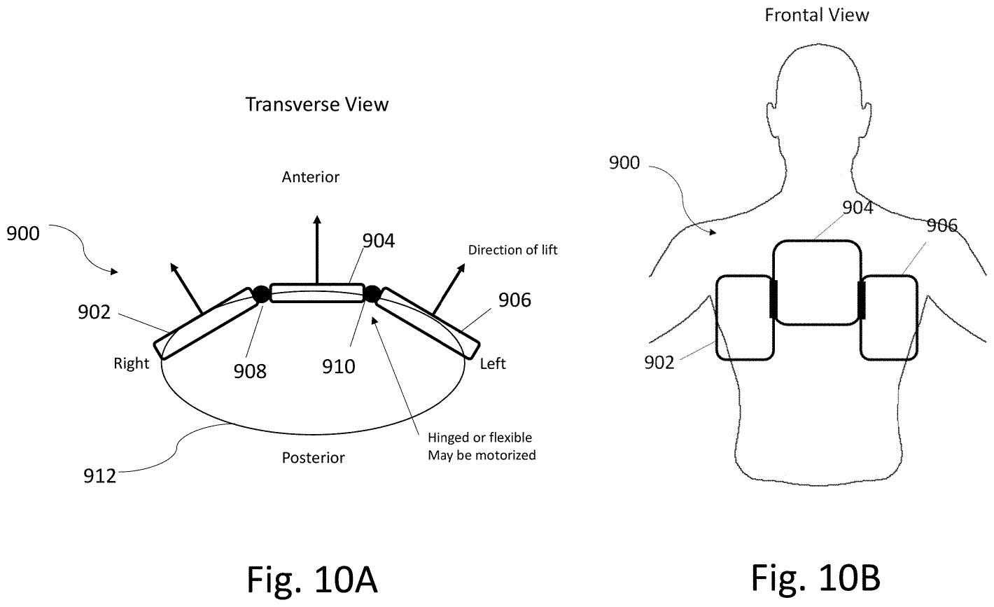

The negative pressure ventilation mechanism 102 can further include a contact surface 116 configured to adhere to a patient's chest. The contact surface 116 can be disposed on or removably attached to the piston 106 or the contact member 154. The contact surface 116 can include a suction cup, adhesive, or any other means of adhering to a patient's chest. Additionally or alternatively, contact surface 116 can be a separate accessory feature. For example, contact surface 116 can include a large adhesive wound dressing that can be placed on the patient's chest/abdomen to allow better suction cup or piston adhesion. In some examples, contact surface 116 can be disposable after each use. Additional or alternative examples of contact surface 116 and contact member 154 are described below with reference to FIGS. 10-12.

The negative pressure ventilation mechanism 102 further can include retention structure 108 configured to at least partially encircle a patient's torso. Retention structure 108 can include one or more legs 110 and/or a support portion 112 configured to be placed underneath a patient 114. Support portion 112 can include at least one of a back plate, a stretcher, a bed, or a cot, or any combination thereof. In some examples, the support portion 112 is configured to maintain contact with a patient's torso. For example, in some examples the support portion 112 can include at least one of an adhesive or suction cup configured to adhere to at least one of a patient's back or one or more straps configured to retain a patient torso on the support portion 112.

The negative pressure ventilation mechanism 102 may include a driver 118 configured to drive the negative pressure ventilation mechanism 102 to cause the negative pressure ventilation mechanism 102 to perform expansions, compressions, or a combination of expansions and compressions of at least one of a chest or an abdomen of patient 114. The controller 104, as will be discussed in more detail below, provides instructions to the negative pressure ventilation mechanism 102 to operate the negative pressure ventilation mechanism 102 at a number of different rates, waveforms, depths, heights, duty cycles or combinations thereof that change over time. Example chest and/or abdomen manipulation instructions or protocols include a series of expansions and/or a series of expansions with small expiration compressions. Additionally or alternatively, exemplary protocols may include a series of expansions with intermittent releases in which a manipulation element is moved away from or otherwise released from the patient's chest and/or abdomen to a retreat position that allows for natural ventilation of a patient's chest, such as a gap or space between the surface of the manipulation element and the patient's chest, without applying any force or active compression or decompression on the patient's chest. During these “releases” between chest and/or abdomen manipulation, the patient's chest and/or abdomen may expand from natural ventilation whether spontaneous or ongoing, or from manual, rescuer administered ventilation from manual ventilation (“rescue breathes”) or from a ventilation device like a ventilator. Additionally or alternatively, exemplary protocols may include a CPR protocol including a series of compressions for CPR and/or a series of expansions for ventilation and compressions for CPR. For example, an exemplary protocol may include alternating an expansion for ventilation and a compression for CPR. In yet another alternative, the alternation in ventilation and compression occurs not at a 1:1 ratio but at a ratio of one expansion for every 5 to 30 compressions to each ventilation or 1 to 30 ventilations to each compression. In addition, the compression rate may be temporarily paused to allow sufficient time for expansion before resumption.

Additionally or alternatively, an exemplary protocol may include a high frequency ventilation protocol including a series of high frequency compressions of a patient's chest and/or abdomen at a shallower depth. Additionally or alternatively, the high frequency ventilation protocol can include a series of high frequency expansions of a patient's chest. For example, the frequency in both exemplary high frequency ventilation protocols may be greater than three Hz, such as between 3-15 Hz. The compressions may be performed at a depth less than 4 centimeters or 1.5 inches. In some examples, the compression depth is between 1-4 centimeters or 0.5 to 1.5 inches. Alternatively, in some examples, a high frequency ventilation protocol may include alternating between compressing and expanding a patient's chest. Such alterations are not limited to a 1:1 ratio, and any number of high frequency ventilation compressions may be performed between any number of high frequency ventilation expansions.

Additionally or alternatively, an exemplary protocol may include a high frequency interposed or superimposed by one or more cycles of CPR. In yet another exemplary protocol, the exemplary protocol may include alternating a high frequency ventilation protocol and a negative pressure ventilation protocol. Another exemplary protocol may include alternating between a high frequency ventilation protocol, a negative pressure ventilation protocol, and CPR cycles.

The controller 104 may include a processor 120, which may be implemented as any processing circuitry, such as, but not limited to, a microprocessor, an application specific integration circuit (ASIC), programmable logic circuits, etc. The controller may further include a memory 122 coupled with the processor 120. Memory can include a non-transitory storage medium that includes programs 124 configured to be read by the processor 120 and be executed upon reading. The processor 120 is configured to execute instructions from memory 122 and may perform any methods and/or associated operations indicated by such instructions. Memory 122 may be implemented as processor cache, random access memory (RAM), read only memory (ROM), solid state memory, hard disk drive(s), and/or any other memory type. Memory 122 acts as a medium for storing data 126, such as event data, patient data, etc., computer program products, and other instructions.

Controller 104 may further include a communication module 128. Communication module 128 may transmit data to a post-processing module 130. Alternately, data may also be transferred via removable storage such as a flash drive. While in module 130, data can be used in post-event analysis. Such analysis may reveal how the medical device was used, whether it was used properly, and to find ways to improve future sessions, etc.

Communication module 128 may further communicate with other medical device 132. Other medical device 132 can be a cardiopulmonary resuscitation (CPR) device, defibrillator, a monitor, a monitor-defibrillator, a ventilator, a capnography device, or any other medical device. Communication between communication module 128 and other medical device 132 could be direct, or relayed through a tablet or a monitor-defibrillator. Therapy from other device 132, such as CPR or defibrillation shocks, can be coordinated and/or synchronized with the operation of the medical device. For example, negative pressure ventilation mechanism 102 may pause the expansions for delivery of a defibrillation shock or CPR.

The controller 104 may be located separately from the negative pressure ventilation mechanism 102 and may communicate with the negative pressure ventilation mechanism 102 through a wired or wireless connection 134. The controller 104 also electrically communicates with a user interface 136. As will be understood by one skilled in the art, the controller 104 may also be in electronic communication with a variety of other devices, such as, but not limited to, another communication device, another medical device, etc.

The negative pressure ventilation mechanism 102 may include one or more sensors configured to transmit information to controller 104. For example, negative pressure ventilation mechanism 102 can include a physiological parameter sensor 138 for sensing a physiological parameter of a patient and to output a physiological parameter sensor signal 140 that is indicative of a dynamic value of the parameter. The physiological parameter can be an Arterial Systolic Blood Pressure (ABSP), a blood oxygen saturation (SpO2) or plethysmograph, a ventilation measured as End-Tidal CO2 (ETCO2) or capnography waveform, invasive blood pressure data, a temperature, a detected pulse, inspired oxygen (O2), air flow volume, etc. In addition, this parameter can be what is detected by defibrillator electrodes that may be attached to patient, such as electrocardiogram (ECG) and transthoracic impedance.

Additionally or alternatively, the negative pressure ventilation mechanism 102 can include a height sensor 142 configured to sense the height of the patient's chest and/or abdomen and to output a height signal 144, which is indicative of the resting height of the patient's chest. Additionally or alternatively, the controller 104 can receive the height signal 144. The received or measured height signal can be used by the controller 104 to calculate a reference position, also referred to as a start position, for the negative pressure ventilation mechanism 102. Additionally or alternatively, the controller 104 can receive a height signal 144 from the height sensor 142 and calculate expansion or compression distance of piston 154 as a percentage of the reference/start position. Additionally or alternatively, the chest compression mechanism can include a movement sensor 146 configured to sense movement of one or both of the patient's chest or abdomen and to output a movement signal 148, which may indicate ventilation movement of the patient's chest and/or abdomen. Additionally or alternatively, the negative pressure ventilation mechanism 102 can include a patient contact member sensor 150 configured to sense the type or configuration of the patient contact member and to output a patient contact member signal 152, which is indicative of whether the patient contact member is configured for compressions or expansions of a patient's chest.

Operations of the medical device 100 may be effectuated through the user interface 136. The user interface 136 may be external to or integrated with a display. For example, in some examples, the user interface 136 may include physical buttons located on the medical device 100, while in other examples, the user interface 136 may be a touch-sensitive feature of a display. The user interface 136 may be located on the medical device 100, or may be located on a remote device, such as a smartphone, tablet, PDA, and the like, and is also in electronic communication with the controller 104. In some examples, controller 104 can receive a rate, a waveform, and/or depth input from the user interface 136 and, responsive to the rate, the waveform, and/or depth input, cause the negative pressure ventilation mechanism 102 to move to adjust the rate, waveform, and/or depth of the expansions and/or compressions during a session. Responsive to a pause input, the controller 104 can move and retain the negative pressure ventilation mechanism 102 in a retreat position for a preset amount of time or negative pressure ventilation or high frequency ventilation can be manually restarted by a rescuer taking an additional action, such as releasing a pause button or otherwise inputting an instruction received by the controller 104 to resume chest and/or abdomen expansions.

During a negative pressure ventilation session of expansions, controller 104 can generate or receive an instruction (either pre-programmed or customized based on any parameters or sensor input or other data) to drive the negative pressure ventilation mechanism 102 to perform at least two cycles of negative pressure ventilation. Each cycle can include the negative pressure ventilation mechanism moving from a reference position away from the patient's chest and/or abdomen to an expansion position to expand a chest and/or abdomen of a patient to generate negative pressure ventilation. The expansion position may be up to 39 millimeters above the reference position. The reference position, also referred to as initial or start positon, can be a specific and pre-defined position or can be calculated or estimated based on sensed input or other patient and/or rescuer data. The same or a subsequent instruction can also drive the negative pressure ventilation mechanism 102 to return to the reference position from the expansion position. In some examples, the same or a subsequent instruction can also drive the negative pressure ventilation mechanism 102 to move to an expiatory position. The expiatory position may be only slightly below the reference position, for example 5 to approximately 13 millimeters below the reference position. A series of chest and/or abdomen expansions can include more than two cycles of negative pressure ventilation. Each cycle can vary in terms of rate, waveform, and/or depth of chest and/or abdomen expansion. The lifting force can vary. For example the lifting for can be between 10 Newtons (N) and 200 N.

The controller 104 may also be configured to perform a first cycle of negative pressure ventilation, a first cycle of CPR immediately after the first cycle of negative pressure ventilation, a second cycle of negative pressure ventilation immediately following the first cycle of CPR, and/or a second cycle of CPR immediately following the second cycle of negative pressure ventilation. In other words, the controller 104 may be configured to perform alternating cycles of negative pressure ventilation and CPR. Each of the first and second cycles of CPR may include positioning the piston at the reference position, extending the piston from the reference position to a compression position to compress a chest of a patient, and returning the piston from the compression position to the reference position or above the reference position. In yet another alternative, the alternation in ventilation and compression occurs not at a 1:1 ratio but at a ratio of one expansion for every 5 to 30 compressions to each ventilation or 1 to 30 ventilations to each compression. In addition, the compression rate may be temporarily paused to allow sufficient time for expansion before resumption.

The controller 104 may also be configured to perform at least two cycles of CPR. Each of the at least two cycles of CPR may include positioning the piston at the reference position, extending the piston from the reference position to a compression position to compress a chest of a patient, and returning the piston from the compression position to the reference position or above the reference position.

During a high frequency ventilation session of expansions or compressions, controller 104 can generate or receive an instruction (either pre-programmed or customized based on any parameters or sensor input or other data) to drive the negative pressure ventilation mechanism 102 to perform high frequency ventilations at a rate greater than three Hz. In some examples, the frequency may be between 3-15 Hz.

When expansions are performed during high frequency ventilation, each cycle can include the negative pressure ventilation mechanism 102 moving from a reference position away from the patient's chest and/or abdomen to an expansion position to expand a chest and/or abdomen of a patient to generate negative pressure ventilation. The expansion position may be up to 39 millimeters above the reference position. The reference position, also referred to as initial or start positon, can be a specific and pre-defined position or can be calculated or estimated based on sensed input or other patient and/or rescuer data. The same or a subsequent instruction can also drive the negative pressure ventilation mechanism 102 to return to the reference position from the expansion position. In some examples, the same or a subsequent instruction can also drive the negative pressure ventilation mechanism 102 to move to an expiatory position. The expiatory position may be only slightly below the reference position, for example 5 to approximately 13 millimeters below the reference position. A series of chest and/or abdomen expansions can include more than two cycles of high frequency ventilation. Each cycle can vary in terms of rate, waveform, and/or depth of chest and/or abdomen expansion.

When compressions are performed during high frequency ventilation, each cycle can include the negative pressure ventilation mechanism 102 moving from a reference position toward the patient's chest and/or abdomen to a compression position. The depth of the compression may be between 0.5 and 1.5 inches or between 1 and 4 centimeters. The reference position, also referred to as initial or start positon, can be a specific and pre-defined position or can be calculated or estimated based on sensed input or other patient and/or rescuer data. The same or a subsequent instruction can also drive the negative pressure ventilation mechanism 102 to return to the reference position from the compression position. A series of chest and/or abdomen compressions can include more than two cycles of high frequency ventilation. Each cycle can vary in terms of rate, waveform, and/or depth of chest and/or abdomen expansion.

Changing the duration of a negative pressure ventilation session, high frequency ventilation and/or one or more of waveform, rate, or depth of chest and/or abdomen expansion in a negative pressure ventilation cycle or expansion or compression in a high frequency ventilation cycle can occur manually or as needed and can be triggered either by user prompts, such as pushing a button on the user interface 136 or otherwise inputting data to indicate to the controller 104 to change waveform, rate and/or depth, or automatically by sensed data, such as data automatically sensed by one or more sensors electrically coupled to the medical device, such as one or more patient physiological sensors including but not limited to physiological parameter sensor 138.

The one or more sensors electrically coupled to the medical device, such as the physiological parameter sensor 138, may include an ETCO2 sensor, ECG, invasive blood pressure monitoring, SpO2 sensor, or any combination thereof. The at least one ETCO2 sensor may be located on the medical device 100, or may be located on a remote device or other medical device 132, such as a device for protection of airways such as an endotracheal tube (ET tube), bag valve mask (BVM), naso-pharyngeal airways (NPA) or similar device to maintain an airway, and/or on a mouth piece designed to go over the mouth that may be strapped in place but does not require an ET tube. The SpO2 sensor may be located on the medical device or may be located on a remote device or other medical device 132, such as a pulse oximetry device. The ECG sensor may include defibrillator electrodes including a 2, 3, 5 12, or 15 lead. The ETCO2, ECG and/or SpO2 sensor located on the medical device 100 or a remote device may be in electronic communication with the controller 104. Additionally or alternatively, sensor data from the ETCO2, ECG, invasive blood pressure monitoring, and/or SpO2 sensor may be processed by a standalone ETCO2, ECG, invasive blood pressure and/or SpO2 monitor, a custom mouth piece configured to measure, process, and/or display sensor ETCO2, and/or ECG data, and/or a multi-functional monitor, such as a monitor/defibrillator like the LIFEPAK15 Monitor/Defibrillator sold by Physio-Control, Inc., a subsidiary of Stryker Corp.

The processed data from the ETCO2, ECG, invasive blood pressure, and/or SpO2 sensor can be displayed as CO2, ECG, invasive blood pressure and/or SpO2 levels, absorbance, transmittance, or values. The display can be located on the medical device 100 or may be located on a remote device, such as a smartphone, tablet, PDA, different medical device and the like, and is also in electronic communication with the controller 104 and/or the other medical device that processed the ETCO2, ECG and/or SpO2 sensor data. From the displayed ETCO2, ECG and/or SpO2 value, the medical device operator can determine if the ventilations provide by the medical device 100 are adequate or not. The at least one of ETCO2, capnogram, ECG, invasive blood pressure, SpO2, and/or plethysmograph value can be sufficient for assessment of the effectiveness of the medical device 100 and provides a means for the operator to respond to the situation. The operator response may include one or more of adjusting the position of the medical device 100 with respect to the patient (for example resetting an intubation tube), changing the depth of the expansion cycle, changing the waveform, and changing the rate of the expansion cycle.

The display may include additional information such as fault information, for example if the medical device 100 or the physiological parameter sensor is reporting fault or a general system fault. Fault information may be displayed either visually or audibly, or both visually and audibly. Display information may additionally or alternatively include a warning symbol or sound, for example if ETCO2, ECG, invasive blood pressure and/or SpO2 value is too low and/or out of a predetermined range. In some examples, the color of the physiological parameter sensor may indicate acceptable or unacceptable ranges. For example, green can be used to indicate acceptable data and yellow and/or red can be used to indicate unacceptable or warning level data.

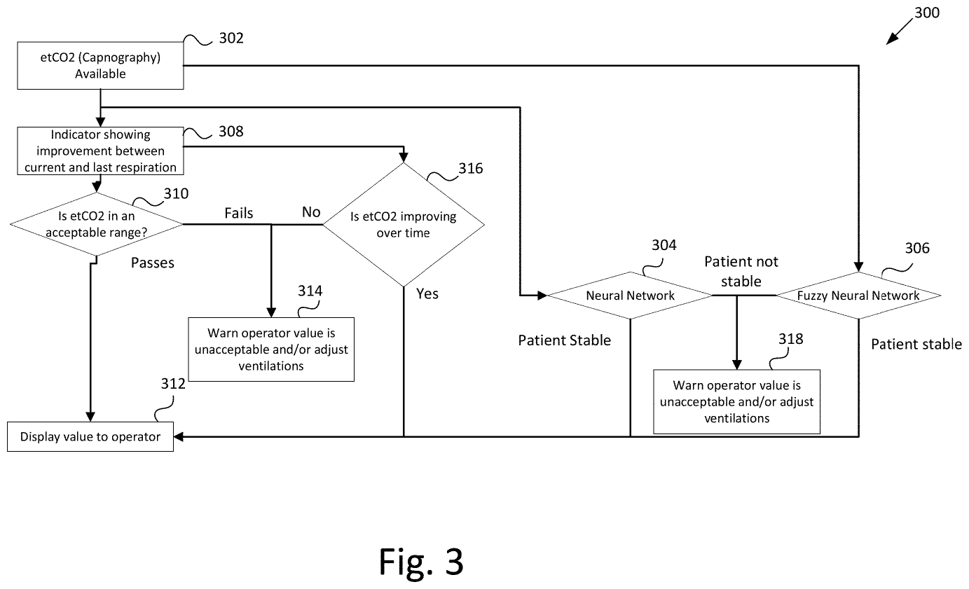

Additionally or alternatively, the medical device 100 may automatically change performance of the negative pressure ventilation cycles or the high frequency ventilation cycles based on sensed ETCO2, capnogram, ECG, invasive blood pressure, SpO2, or plethysmograph values without operator input. The ETCO2, ECG, invasive blood pressure and/or SpO2 sensed data may be processed by controller 104 and/or the processed data may be received by controller 104. The controller 103 can be configured to adjust in real time the rate, duration, waveform, and/or depth of the negative pressure ventilation or the high frequency ventilation based at least in part of the ETCO2, ECG, invasive blood pressure and/or SpO2 values. For example if the ETCO2, ECG, invasive blood pressure and/or SpO2 values are falling, the medical device 100 can automatically increase the rate or increase the depth of the negative pressure ventilation cycle or the high frequency ventilation. The medical device 100 can get real-time feedback from the sensed data and can respond accordingly. The response can be implemented as an automated algorithm. For example, change over time or respirations may be evaluated and the response may be based on defined ranges for each parameter, where the range has set parameters for waveform, rate, depth, and/or duration. Additionally or alternatively fuzzy logic may be used to change the parameters but provide a smooth transition as the value changes from one bin to another. Additionally or alternatively, a neural network can be used, which changes the output parameters based on all input data in real time. Neural Network can be used to determine if patient is likely to survive or if intervention is required. Neural Network can rely on all available data to stream into network. Neural networks can use backward propagation and nodes to make decisions off of inputs. Additionally or alternatively, a Fuzzy Neural Network can be used to stabilize output report determined by a neural network. Sensor data is further described with reference to FIGS. 3-9 below.

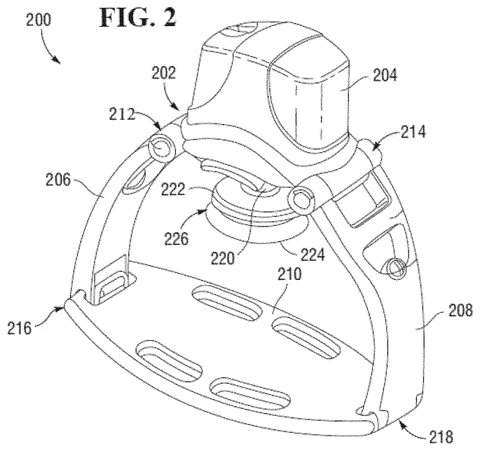

FIG. 2 shows a medical device 200 including a retention structure 202. The retention structure 202 includes a central member 204, a first leg 206, a second leg 208, and a support portion 210 configured to be placed underneath a patient. Central member 204 is coupled with first leg 206 and with second leg 208 via joints 214 and 214, respectively. In addition, the far ends of legs 206, 208 can become coupled with edges 216, 218 of support portion 210. These couplings form the retention structure 202 that retains a patient. In this particular case, central member 204, first leg 206, second leg 208 and support portion 210 form a closed loop, in which the patient is retained.

Central member 204 includes a battery that stores energy, a motor that receives the energy from the battery, and a compression mechanism that can be driven by the motor. The compression mechanism is driven up and down by the motor using a rack and pinion gear. The negative pressure ventilation mechanism includes a chest and/or abdomen manipulation element, such as a piston 220 that emerges from central member 204, and can expand and release the patient's chest. Piston 220 is sometimes called a plunger. Here, piston 220 terminates in a contact member 222 having a contact surface 224. The contact member 222 can include a suction cup 226. Contact member 222 and/or contact surface 224 can be configured to adhere to the patient's chest, for example via a suction cup and/or adhesive surface. In this case the battery, the motor and the rack and pinion gear are not shown, because they are completely within a housing of central member 204.

In some examples, medical device 100 can be configured to have two modes, a first mode for negative pressure ventilation and a second mode for CPR. In other examples, medical device 100 can be figured to have one or more modes, the modes including at least one of negative pressure ventilation, high frequency ventilation, and CPR. The contact member 154 and/or the contact surface 116 may be used in each of the modes. Transition from the modes can be determined automatically by the controller 104 in response to ECG monitoring data received from ECG physiological parameter sensor 138. Additionally or alternatively, the controller 104 can receive instruction from user interface 136 regarding mode selection.

Additionally or alternatively, a first contact member and/or a first contact surface may be configured for use in the first mode. The first contact member and/or first contact surface may be configured to be removable or ejected from piston 106. A second contact member and/or a second contact surface may be configured for used in the second mode. The second contact member and/or second contact surface may be configured to be removable or ejected from piston 106. As noted earlier, the patient contact member sensor 150 can be configured to sense the type or configuration of the contact member and/or contact surface and to output a patient contact member signal 152, which is indicative of whether the patient contact member and/or contact surface is configured for negative pressure ventilation (mode one) or CPR (mode two), to controller 104. Additionally or alternatively, the controller 104 can receive instruction from user interface 136 regarding mode selection. Responsive to receipt of patient contact member signal 152 or instruction from the user interface 136, controller 104 can determine whether to issue instructions for negative pressure ventilation, high frequency ventilation, or CPR based on patient contact member signal 152 or instruction from user interface 136.

FIGS. 3-9 show exemplary flow charts of sensor data. FIG. 3 is a flow chart 300 of an example operation of a medical device including an ETCO2 sensor. This example operation is used when the medical device is on the patient and chest compressions for cardiac output is determined to be unnecessary, and the ECG monitoring is unknown. In this scenario, the patient is not breathing on their own and ventilations are being performed, and ETCO2 data is available.

If ETCO2 data is available in operation 302, then the output of the ETCO2 sensor is transmitted to a fuzzy neural network 304 and a neural network 306. A display may output an improvement of the ETCO2 data between the current and last respiration provided by the medical device 300 in operation 308. If the ETCO2 data is in an acceptable range in operation 310, then the value of the ETCO2 data can be relayed to an operator in operation 312, such as being displayed on a display.

If the ETCO2 data is not in acceptable range, then in operation 314, a warning can be output to an operator, such as a visual or audio alert. Further, in operation 316, it can be determined if the ETCO2 data is improving over time, such as after each respiration, and the display value can be output to the operator in operation 312.

The neural networks in operations 304 and 306 can be used to determine if a patient is likely going to survive or if intervention is required. Neural networks 304 and 306 rely on all available data to stream into the network. As such, they may include more sensor data than just the ETCO2 data in operation 302. The neural networks in operations 304 and 306 use backward propagation and nodes to make decisions off the inputs. The fuzzy neural network in operation 306 is used to stabilize an output report by the neural network in operation 304. If both neural networks determine patient is stable in operations 304 and 306, the data can be output in operation 312. However, if either of the operations 304 and 306 determines that the patient is not stable, a warning may be output to an operator in operation 318.

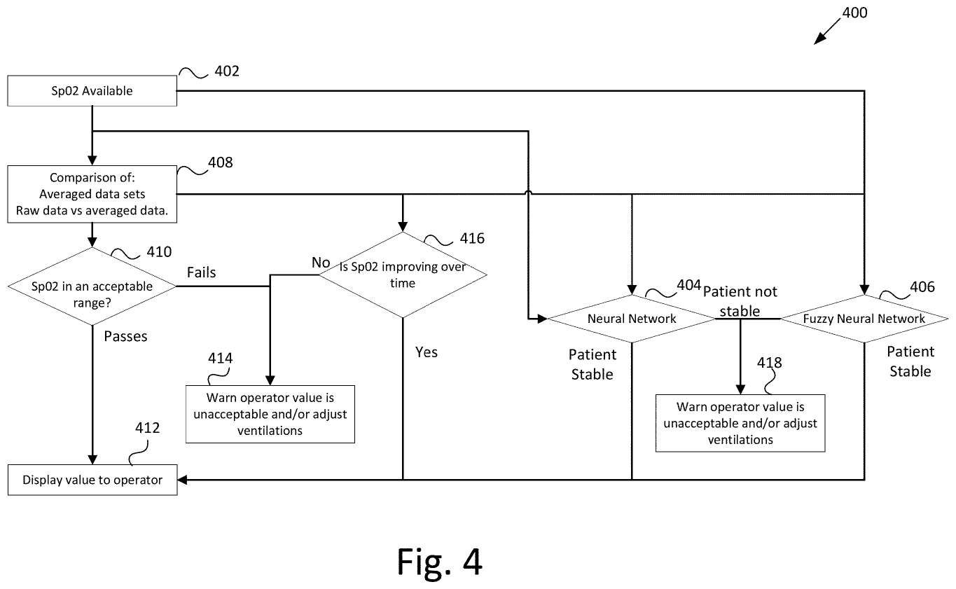

FIG. 4 is a flow chart 400 of an example operation of a medical device including a spO2 sensor. In the example operation of FIG. 4, the medical device is on the patient and chest compressions for cardiac output is determined to be unnecessary, and the ECG monitoring is unknown. In this scenario, the patient is not breathing on their own and ventilations are being performed, but spO2 data is available.

If spO2 data is available in operation 402, then the output of the spO2 sensor is transmitted to a fuzzy neural network 404 and a neural network 406. In operation 408, averaged data sets of spO2 data is compared to raw data. If the spO2 data is in an acceptable range in operation 410, then the value of the spO2 data can be relayed to an operator in operation 412, such as being displayed on a display.

If the spO2 data is not in acceptable range, then in operation 414, a warning can be output to an operator, such as a visual or audio alert. Further, in operation 416, it can be determined if the spO2 data is improving over time, such as after each respiration, and the display value can be output to the operator in operation 412.

The neural networks in operations 404 and 406 can be used to determine if a patient is likely going to survive or if intervention is required. Neural networks 404 and 406 rely on all available data to stream into the network. As such, they may include more sensor data than just the spO2 data in operation 402. The neural networks in operations 404 and 406 use backward propagation and nodes to make decisions off the inputs. The fuzzy neural network in operation 406 is used to stabilize an output report by the neural network in operation 404. If both neural networks determine patient is stable in operations 404 and 406, the data can be output in operation 412. However, if either of the operations 404 and 406 determines that the patient is not stable, a warning may be output to an operator in operation 418.

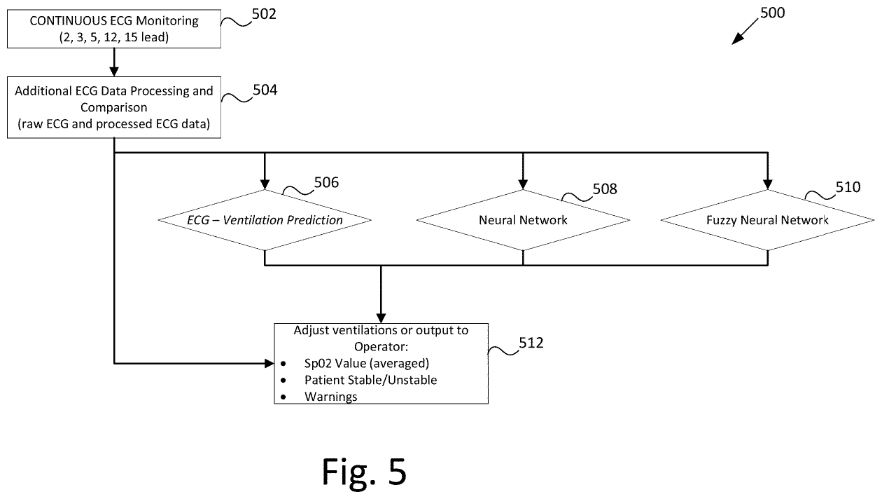

FIG. 5 is a flow chart 500 of an example operation of a medical device including an ECG sensor. In the example operation of FIG. 5 the medical device is on the patient and chest compressions for cardiac output is determined to be unnecessary. In this scenario, the patient is not breathing on their own and ventilations are being performed.

In operation 502, continuous ECG monitoring data is received. In operation 504, the ECG data can be processed and compared to raw data. The output of operation 504 is sent to each of an ECG ventilation prediction operation 506, a neural network operation 508, a fuzzy network operation 510, as well as an operator output operation 512. The operator output operation 512 can output the ECG data, as well as whether the patient is stable or unstable, and any warnings based on the ECG data.

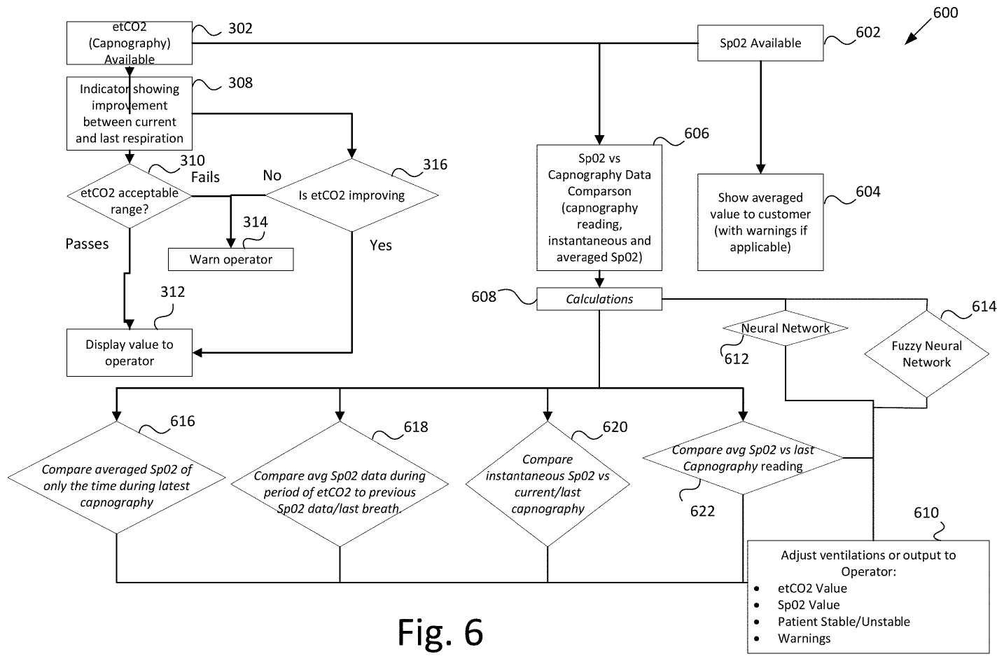

FIG. 6 is a flow chart 600 of an example operation of a medical device including both an ETCO2 sensors and SpO2 sensors. FIG. 6 includes operations similar to those discussed above with respect to FIG. 3 and as such, those operations are given the same reference numbers and are not discussed in detail with respect to FIG. 6.

In this example, both ETCO2 data in operation 302 and spO2 data in operation 602 are available. Both outputs may be displayed to an operator in operations 312 and operations 604. Additional, a controller 104 can compare in the spO2 data and the ETCO2 data in operation 606. In operation 608, a number of different calculations may be performed to determine how a patient is responding to ventilations provided by the medical device 100.

The calculations are illustrated in FIG. 6 as a number of different operations, but each of the output of these calculates can either be displayed to an operator in operation 610 or be input into the neural network operation 612, and/or the fuzzy network operation 614.

For examples, the calculations 610 can include comparing an average spO2 data during the time of the latest capnography in operation 616. Calculations 608 also can include comparing averaged spO2 data during on period of ETCO2 monitoring to previous spO2 data or last breath in operation 618. In operation 620, the instantaneous spO2 value can be compared to a current and/or last capnography. In operation 622, the average spO2 data can be compared to the last capnography reading.

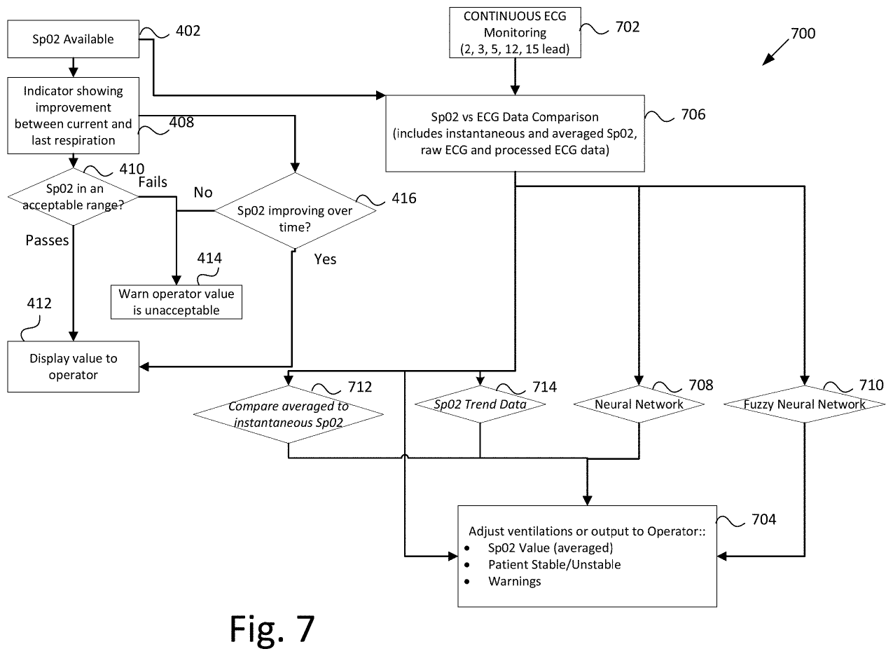

FIG. 7 is a flow chart 700 of an example operation of a medical device including an ECG and SPO2 sensors. FIG. 7 includes operations similar to those discussed above with respect to FIG. 4 and as such, those operations are given the same reference numbers and are not discussed in detail with respect to FIG. 7.

In this example, both ECG data in operation 702 and spO2 data in operation 402 are available. Both outputs may be displayed to an operator in operations 412 and operations 704. Additional, a controller 104 can compare in the spO2 data and the ETCO2 data in operation 706. A number of different calculations may be performed to determine how a patient is responding to ventilations provided by the medical device 100.

The calculations are illustrated in FIG. 7 as a number of different operations, but each of the output of these calculates can either be displayed to an operator in operation 704 or be input into the neural network operation 708, and/or the fuzzy network operation 710. For examples, the calculations can include comparing an averaged to instantaneous spO2 value in operation 712 or determining spO2 trend data in operation 714.

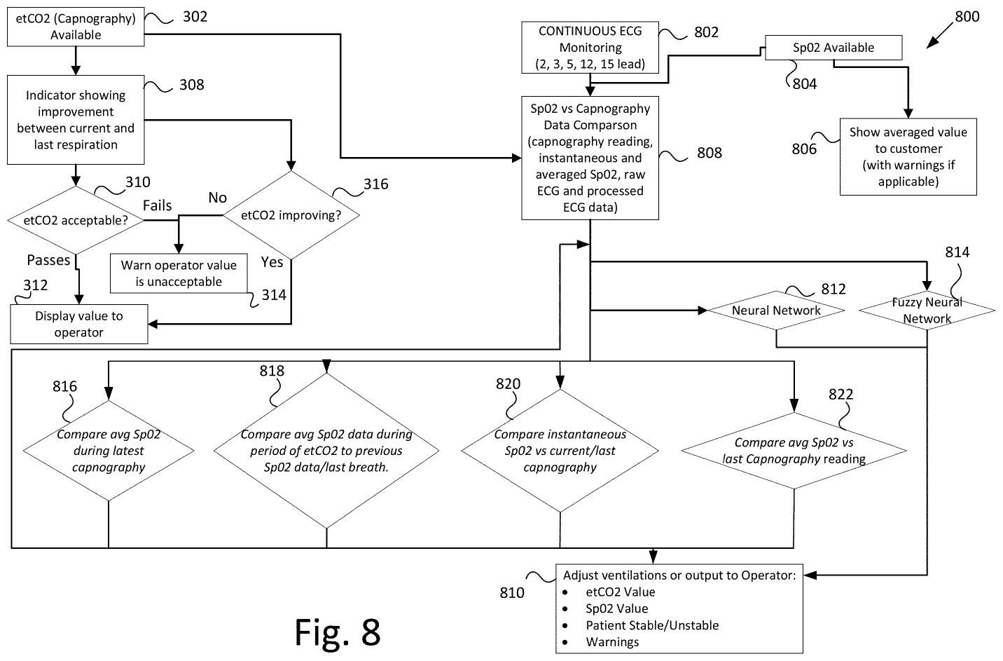

FIG. 8 is a flow chart 800 of an example operation of a medical device including an ETCO2, ECG and SPO2 sensors. FIG. 6 includes operations similar to those discussed above with respect to FIG. 3 and as such, those operations are given the same reference numbers and are not discussed in detail with respect to FIG. 6.

In operations 802 and 804, continuous ECG monitoring data is received and spO2 data is received, respectively. In operation 806, averaged spO2 data can be displayed to an operator, with any relevant warnings, similar to those described above in FIG. 4. In operation 808, spO2, ETCO2 and ECG data can be compared. A number of calculations can be performed or output to an operator in operation 810 or inputs to either the neural networks in operations 812 and 814.

The calculations may include, for example, comparing average spO2 data of only a time during a latest ETCO2 in operation 816. In operation 818, averaged spO2 data during one period of ETCO2 monitoring can be compared to previous spO2 data and/or the last breath. In operation 820, the instantaneous spO2 data can be compared to current versus last capnography. In operation 822, the averaged spO2 vs last capnography reading can be compared. Further calculations may also be performed that are not explicitly shown in FIG. 8 but which would be beneficial to an operator.

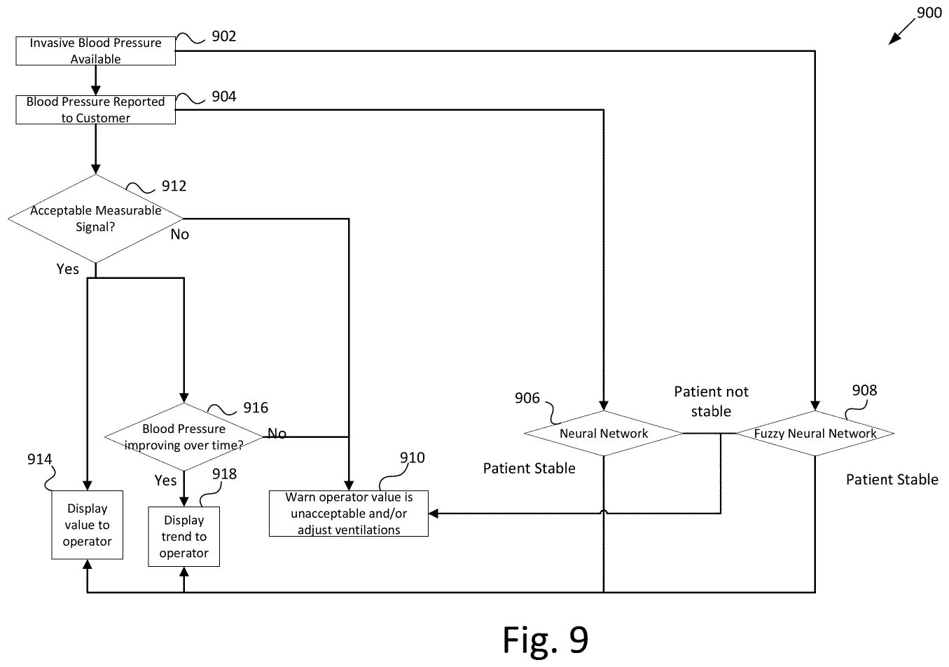

FIG. 9 is a flow chart 900 of an example operation of an example of a medical device including invasive blood pressure monitoring sensor data. In this example, in operation 902 it is determined that invasive blood pressure is available. The blood pressure can be reported to an operator in operation 904, as well as sent operations 906 and 906 to process the data in a neural network and a fuzzy neural network, respectively. Similar to other examples above, operations 906 and 908 can determine if a patient is stable and warn an operator in operation 910 if the patient is not stable.

In operation 912, a controller 104 can determine if the measured blood pressure is acceptable. If yes, in operation 914, the blood pressure value can be displayed. If no, then in operation 916, an operator can be warned. Further, if the blood pressure is acceptable, it can be checked if spO2 is improving over time or after each respiration performed by the medical device 100 in operation 916. Then trend can be displayed to an operation in operation 918.

In each of the example operations discussed above with respect to FIGS. 3-9, a controller 104 in medical device 100 can not only display values to an operator and/or output audio or visual information to the operator, but can also adjust the waveform, rate, depth, and/or duration of the ventilations, as discussed above.

FIGS. 10A, 10B, 11 and 12 are additional or alternative examples of contact member 154 and/or a contact surface 116. For example FIGS. 10A and 10B show contact surface 900 including a first, second, and third plate 902, 904, 906 that can be fixed or flexibly attached to one another at connection areas 908 and 910. Additionally or alternatively, connection areas 908 and 910 may be motorized to mechanically move one or more of first, second, and third plate 902, 904, and 906. One or more of first, second, and third plate 902, 904, 906 may include adhesive configured to adhere to the patient's chest and/or abdomen. Additionally or alternatively, first, second, and third plate 902, 904, 906 may be configured for attachment to the manipulation element, such as a piston, and/or a contact member, such as a suction cup attached to the piston. Contact surface 900 may further include a strap 912 configured to wrap around the patient's torso. Additionally or alternatively, there may be a fourth plate connected with similar mechanism as 902, 904, and 906 to facilitate expansion of larger areas around the chest such as a plate over the abdomen.

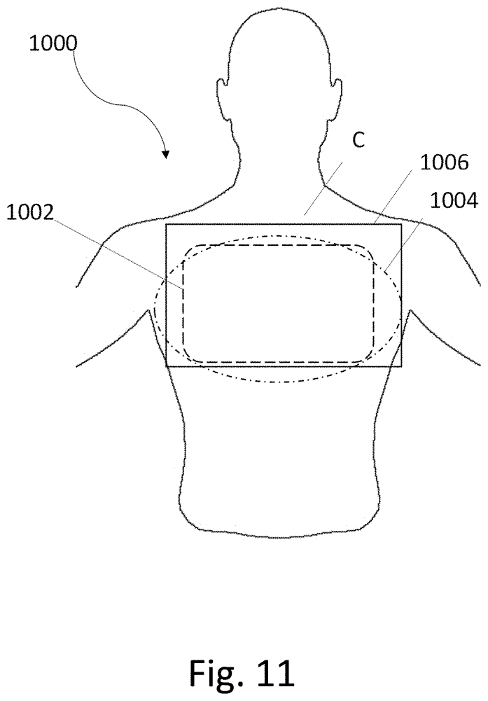

FIG. 11 shows contact surface 1000 configured to provide external negative pressure to stabilize a chest C. Specifically contact surface 1000 includes a resilient member 1002 configured to cover a substantial portion of the patient's chest. Additionally or alternatively, contact surface 1000 can be configured to cover a substantial portion of the patient's chest and abdomen or a substantial portion of the patient's abdomen alone. Resilient member 1002 may be configured to provide some structural support to the chest while still being flexible. For example resilient member 1002 can include flexible foam. An interface material 1004 may be disposed between the chest and resilient member 1002 to protect the patient's skin. The resilient member 1002 may be attached to the chest via an adhesive material 1006 such as tape. The contact surface 1000 may be configured to adhere or attached to a contact member such as a suction cup or a manipulation element such as a piston.

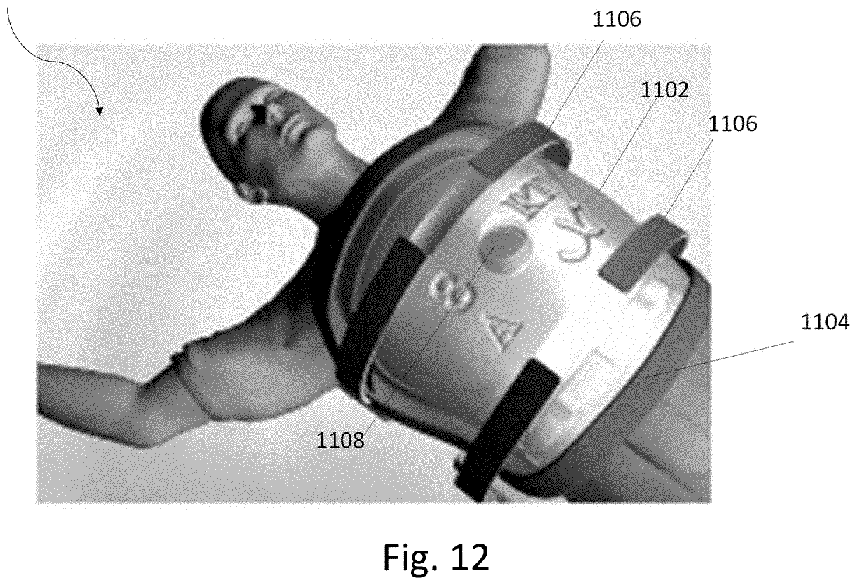

FIG. 12 shows an alternative contact surface 1100 including a hard shell 1102 configured to substantially cover the chest and abdomen of a patient and a seal 1104, such as a foam seal, that maintains an airtight fit around the patient. Hard shell 1102 is designed to fit within the retention structure of medical device 100. Alternatively and/or additionally, hard shell 1102 is designed to replace the retention structure of medical device 100 thereby performing a dual function. Shell 1102 may be include a resilient material such as plastic. One or more straps 1106 can be used to hold the shell 1102 in place. One example of shell 1102 is manufactured by United Hayek Company under the brand name Biphasic Cuirass Ventilation. The shell 1102 can include a contact point 1108. Contact point 1108 can be configured to releasably engage with a piston and/or a contact member. Piston movement can be used to create negative pressure, causing air to rush in through the nose and mouth and into the lungs.

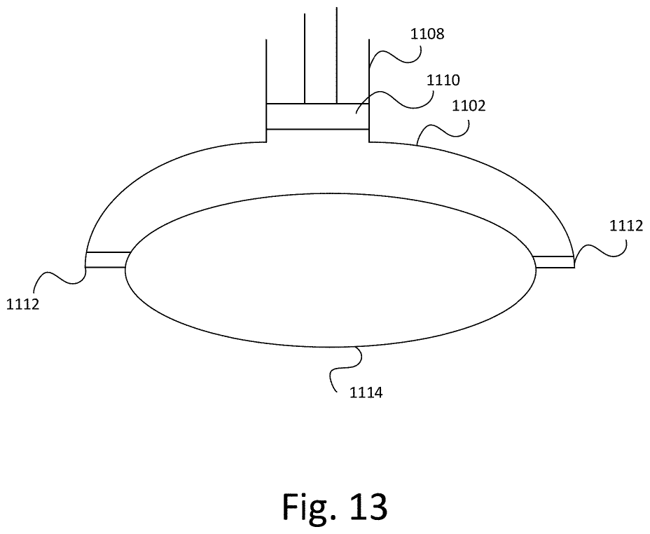

In one example, as shown in FIG. 13 contact point 1108 may be a cylindrical opening into the resilient shell 1102 with diameter and length determined by the volume of air to be displaced to provide negative pressure ventilation. Contact surface 1100 can work in conjunction with medical device 200 by swapping out the suction cup 222 with a more rigid plunger or suction cup 1110 designed to create an airtight or nearly airtight seal with cylinder 1108. Piston 220 of medical device 200 would then displace the appropriate volume of air to perform negative pressure ventilation by moving the rigid plunger or suction cup 1110 back and forth through the cylinder 1108. Gaskets 1112 may be provided to create a seal against patient 1114. Although not shown, one or more straps, similar to those shown in FIG. 12 may be used to hold the shell 1102 to the patient 1114.

The one or more straps allow for the application of positive pressure, as well as allow compressions similar to load-distributing band compressions. A dual piston system could allow compressions and ventilations at the same time, but having dual pistons produce from the center of the suction cup down to the chest of the patient.

Other examples of contact member 154 and/or contact surface 116 can include a system of pulleys, wires, and/or motors that comprise part of the piston footprint configured to lift/expand the chest and/or abdomen for at least one of negative pressure ventilation or high frequency ventilation. These pulleys or motors can be configured to help facilitate lateral expansion of the thorax in coordination with chest lifting to further enhance the volume of negative pressure ventilation. The force applied by more lateral aspects of the contact member and/or contact surface may be lifted with equal or different force than the central aspect of the contact member and/or contact surface. The contact member and/or contact surface may include shape memory alloys or electroactive polymers that may facilitate lateral chest wall lifting without additional fixtures such as pulleys.

Additionally or alternatively, medical device 100 can include more than one chest and/or abdomen manipulation element 106. For example, medical device 100 can include a central piston and two lateral pistons configured to help facilitate lateral expansion of the thorax. Additionally or alternatively, negative pressure ventilation can include an inhalation step including lifting the chest and/or abdomen using a piston with suction cup or adhesive and an exhalation step including a slight compression from a load distributing band type chest compression device. Additionally or alternatively, negative pressure ventilation can include an inhalation step including lifting the chest only. Additionally or alternatively, negative pressure ventilation can include an inhalation step including lifting the abdomen only. Additionally or alternatively, negative pressure ventilation can include an inhalation step including synchronous lifting the chest and abdomen. Additionally or alternatively, negative pressure ventilation can include an inhalation step including asynchronous lifting the chest and abdomen.

Additionally or alternatively, the medical device includes or is connected to a monitor and a drop in blood O2 saturation, or rise in CO2 in the exhaled breath may lead to automatic increase in tidal volume and/or ventilation rate. Additionally or alternatively, detection of near depletion of exhaled O2 may lead to an increase in tidal volume and/or ventilation rate. Additionally or alternatively, an exhaled CO2 below a threshold can lead to the device decreasing tidal volume and/or ventilation rate.

Additionally or alternatively, the medical device can include or be connected to a monitor for ECG (and/or other physiological parameters) or accepts user input to quickly transition from ventilation mode to CPR mode in the event that a patient arrests during care. In one example this transition includes removal or ejection of a first contact member and/or a first contact surface that may be configured for negative pressure ventilation or high frequency ventilation from the piston over the center of the chest.

In some examples, the distance the chest and/or abdomen is lifted or expanded is a fixed number. In other examples, the chest and/or abdomen lift or expansion is determined by force applied and/or relative distance from the initial/neutral position. Additionally or alternatively, a medical device may adjust lift and/or compress rates as well as waveform not just lift/compress distance and frequency of ventilation.

Target ventilation volumes for the medical device may include 4-20 ml/kg, respiratory rates of 6-20 breaths per minute for adults and 20-35 for pediatrics. The medical device may be configured for use with children. Pediatric versions might have smaller target tidal volumes and higher ventilation rate that can be achieved by moving the plunger less distance and faster or more frequently. Contact members and support structures as described in medical device 100 may also be pediatric specific in specifications such as size. A user interface may allow adjustment of tidal volume (i.e., change lifting force or distance) and ventilation rate. The user interface may also have a pediatric mode button than allows rapid switching between standard adult therapy and standard pediatric therapy. The user interface may be removable so it's not in the way when the device is used for CPR. The default parameters of the device in both pediatric and adult mods may be adjustable through the user interface or wireless user interface.

Additionally or alternatively, the medical device could receive feedback from airway monitoring devices and automatically adjust the tidal volume, for example, if the measured pressures are high but flow rate is low the device could interpret that there is obstructive pulmonary disease and adjust tidal volumes and rates accordingly. This may be done by decreasing tidal volume by decreasing the lift and/or compress speed.

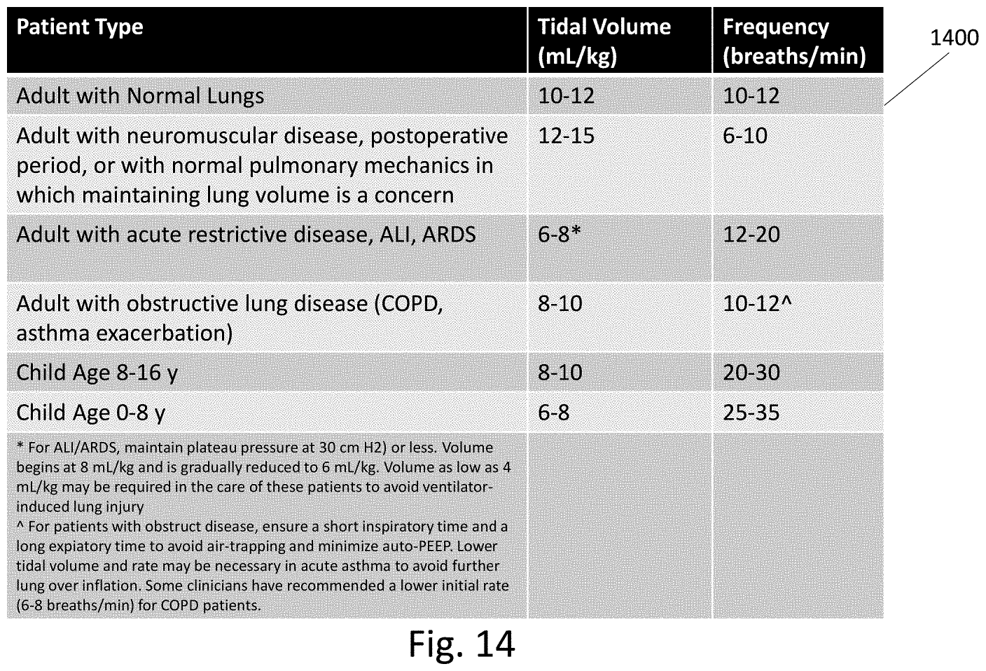

FIG. 14 is a table 1400 of starting points for tidal volumes and ventilation rates for various ages and conditions (Egan's Fundamentals of Respiratory Care 8th Edition by Wilkins Stroller and Scanland) and the device would enable reaching these values.

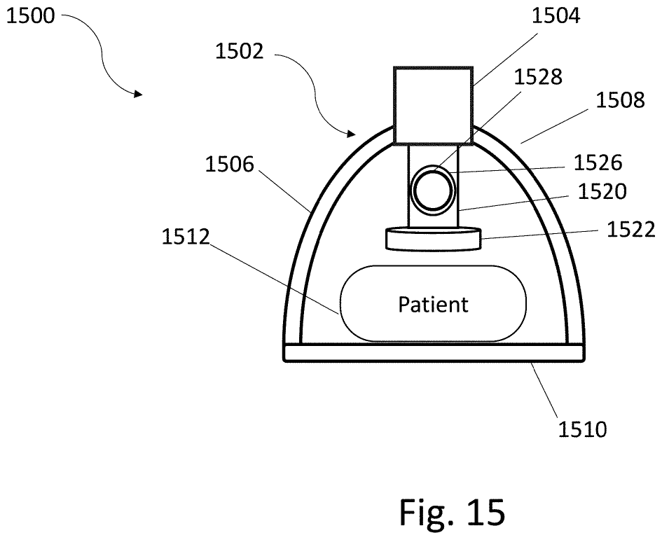

FIG. 15 shows a medical device 1500 including a retention structure 1502. The retention structure 1502 includes a central member 1504, a first leg 1506, a second leg 1508, and a support portion 1510 configured to be placed underneath a patient. Central member 1504 is coupled with each of the first leg 1506 and the second leg 1508. In addition, the far ends of legs 1506, 1508 can become coupled with edges of support portion 1510. These couplings form the retention structure 1502 that retains a patient. In this particular case, central member 1504, first leg 1506, second leg 1508 and support portion 1510 form a closed loop, in which the patient 1512 is retained.

Central member 1504 includes a battery that stores energy, a motor that receives the energy from the battery, and a compression mechanism that can be driven by the motor. The compression mechanism is driven up and down by the motor, such as, for example, by using a rack and pinion gear or any other linear actuator. The central member 1504 includes a chest and/or abdomen manipulation element, such as a piston 1520 that emerges from central member 1504, and can compress the patient's chest. Piston 1520 is sometimes called a plunger. Here, piston 1520 terminates in a contact member 1522. In this case the battery, the motor and the rack and pinion gear are not shown, because they are completely within a housing of central member 1504.

The piston 1520 may include a compartment or slot 1526 for receiving a ventilation bag 1528. Rather than or in addition to pulling the chest of the patient 1512 upward to provide a negative pressure ventilation and/or decompression, a ventilation may be provided by compressing the ventilation bag 1528 when the piston 1520 is retracted from the chest of the patient 1512.

That is, in this example medical device 1500, during CPR a ventilation bag 1528 may be placed in the slot 1526 within the piston 1520. When the controller extends the piston 1520 during a chest compression, no breath is given because the ventilation bag 1528 is not compressed. When the piston 1520 retracts, either by releasing the chest or by performing a decompression, the ventilation bag 1528 can be compressed by the piston 1520 rising toward the central member 1504. The ventilation bag 1528 can then be compressed against the central member 1504 when contained in the slot 1526. When the ventilation bag 1528 is compressed by the central member 1504 when the piston 1520 is retracted, a ventilation is provided to the patient 1512.

A controller 104 in the central member can operate the medical device 1500 under a preset program for providing both compressions and ventilations to the patient 1512 so that the controller 104 can control a timing of the piston 1520 as well as the amount of extension and retraction. That is, the controller 104 can control how often the piston 1520 compresses the chest and how often the piston 1520 retracts to compression the ventilation bag 1528.

The controller 104 may control the piston 1520 to retract to different positions from the patient, depending on whether a ventilation is to be provided. For example, during normal chest compressions without ventilations, the controller 104 can control the piston 1520 to retract to a first position that does not cause the ventilation bag 1528 to be compressed during chest compressions only. When a ventilation is to be given, the controller 104 can cause the piston 1520 to retract to a second position such that the ventilation bag 1528 can be compressed against the central member 1504.

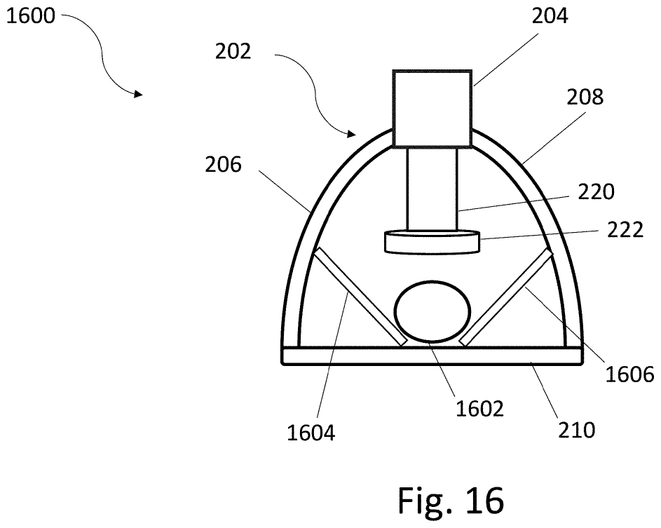

FIG. 16 shows another example of a medical device 1600. In this example, the medical device 1600 may be similar to medical device 200 and similar components are given the same reference numbers as those in FIG. 2 and not discussed further.

The medical device 1600 can perform compressions and/or negative pressure ventilations according to examples discussed above. However, in some examples, a ventilation only mode may be selected by an operator and the medical device 1600 can be structured or modified to provide compressions to a ventilation bag 1602 to compress the bag to provide automatic ventilations to a user when chest compressions are not required.

Support segments 1604 and 1606 may attach to the legs 206 and 208 of the medical device 1600 by use, for example, of a strap or any other attachment means. In some examples, each of the legs 206 and 208 may have one or more attachment points to attach the support segments 1604 and 1606. The support segments 1604 and 1606 may attach to a number of different points of each of the legs 206 and 208 which can accommodate a variety of different ventilation bag 1602 sizes and volumes. For example, the space between the support segments 1604 and 1606 on the backboard can be adjusted by adjusting the attachment points to the legs 206 and 208. In some examples, the support segments 1604 and 1606 may be stored within or be a portion of the support portion 210.

As seen in FIG. 16, the support segments 1604 and 1606, when attached to the legs 206 and 208, have a “V”-shape to hold or cradle the ventilation bag 1602 as the piston 220 compresses the ventilation bag 1602. A controller 104 can operate the piston 220 at a programmed rate for compressing and releasing the ventilation bag 1602 during a ventilation only mode.

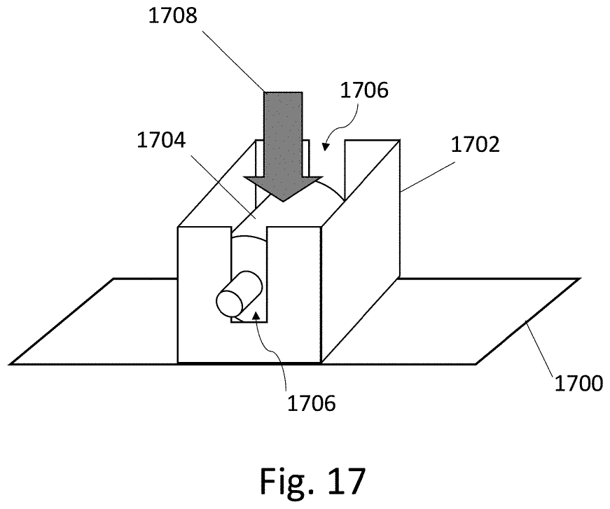

FIG. 17 shows an alternative backboard 1700 which may be used instead of the support portion 210. The patient support portion 210 may be disconnected from the medical device 200 and the backboard 1700 may be attached to the legs 206 and 208. The backboard 1700 can include a container 1702. In some examples, the container 1702 may be strapped or otherwise attached to the patient support portion 210, rather than providing a secondary backboard with the container 1720 already attached. The container 1702 may attach to the patient support portion 210 by straps or any other fasteners, such as screws, suction cups, adhesive, etc.

The backboard 1700 and/or container 1702 itself may be used with any chest compressions mechanism device that has the capability of compressing the bag. The chest compression device can have a ventilation only mode programmed, and a user may use the chest compression device as an automatic ventilator in situations where chest compression for a patient is not necessary. A controller 104 of the chest compression device can be controlled to extend a chest compression member to compress a ventilation bag 1704 within the container 1702 to provide automatic ventilations to a patient. The chest compression member would compress the ventilation bag 1704 in the direction of arrow 1708.

The container 1702 can be structured to receive the ventilation bag 1704. That is, the container 1702 has an opening to receive the ventilation bag 1704. The container 1702 may also have slots 1706 on opposite sides of the container to receive values, tubes, and/or sensors that may connect to the patient and/or a gas supply for ventilation. Rather than slots 1706, in some examples, two of the sides of the container 1702 may be completely open to accommodate the various valves, tubes, and/or sensors. The inclusion of valves on the ventilation bag 1704 can reduce the risk of barotrauma.

The container 1702 may come in a number of different sizes so that an operator can select an appropriate container size based on the size of the ventilation bag 1704 and attach the container 1702 to a patient support portion 210. Additionally or alternatively, the container 1702 may come with additional packing material to make the container 1702 smaller to securely hold smaller ventilation bags 1704. Additionally or alternatively, the container 1702 may have a height adjustable bottom structured to move the ventilation bag 1704 up or down, as needed.

For example, the container 1702 may include an adjustable platform (not shown), either mechanically or electrically, to raise or lower the ventilation bag 1704. For example, the insides of the container 1702 may contain slots or any other attachment type to hold a platform at a desired height for the ventilation bag 1704. Smaller ventilation bags 1704 may be contained a higher height than a larger ventilation bag 1704. In other examples, a block may be placed within the container 1702 to raise the height of the ventilation bag 1704.

Based on the ventilation bag 1704 size, height, and the compression depth of the piston 220, the ventilation volume can be adjusted. In some examples, an analog scale, such as on calipers, could be included within the container 1702 to provide more accurate height adjustment to achieve a calibrated ventilation volume.

Although a rectangular container 1702 is illustrated in FIG. 17, examples of the disclosure are not limited to this shape. Container 1702 may be any shaped container to hold a ventilation bag 1704 in place so that the piston 210 can compress the bag.

For purposes of this description, certain aspects, advantages, and novel features of the examples of this disclosure are described herein. Features, integers, characteristics, compounds, chemical moieties or groups described in conjunction with a particular aspect, example of the invention are to be understood to be applicable to any other aspect, example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C,” or “A, B, and C.”

As used herein, the term “coupled” generally means physically coupled or linked and does not exclude the presence of intermediate elements between the coupled items absent specific contrary language.

What is claimed is:

1. A medical device, comprising:
a retention structure for at least partially encircling a patient's body, the retention structure including a central member and a support portion configured to be placed underneath a patient;
a piston extending from the central member;
a driver coupled to the piston configured to retract and extend the piston;
a patient contact member attached to the piston, the patient contact member configured to adhere to the patient's body; and
a controller configured to cause the driver during a session to perform at least two cycles of negative pressure ventilation, each of the at least two cycles of negative pressure ventilation including:
position the piston at a reference position;
retract the piston from the reference position to an expansion position to expand a chest of a patient to generate negative pressure ventilation; and
return the piston from the expansion position to the reference position.

2. The medical device of claim 1, wherein the at least two cycles of negative pressure ventilation further include:
moving the piston from the reference position to an expiatory position; and
returning the piston from the expiatory position to the reference position.

3. The medical device of claim 1, wherein the patient contact member includes an adhesive surface configured to adhere to the patient's body.

4. The medical device of claim 1, wherein the patient contact member includes a suction cup configured to adhere to the patient's body.

5. The medical device of claim 1, further comprising:
a physiological parameter sensor for sensing a physiological parameter of a patient and to output a physiological parameter sensor signal that is indicative of a dynamic value of the parameter, the physiological parameter sensor configured to transmit information to the controller.

6. The medical device of claim 5, wherein the controller is configured to adjust one or more of rate, waveform, or depth of the at least two cycles of negative pressure ventilation based at least in part on the physiological parameter sensor signal.

7. The medical device of claim 5, wherein the controller is configured to change to a cardiopulmonary resuscitation (CPR) mode based at least in part on the physiological parameter sensor.

8. The medical device of claim 1, wherein the controller is further configured to perform at least two cycles of cardiopulmonary resuscitation (CPR), each of the at least two cycles of CPR including:
position the piston at a reference position;
extend the piston from the reference position to a compression position to compress a chest of a patient; and
return the piston from the compression position to the reference position or above.

9. The medical device of claim 1, further comprising:
a user interface configured to transmit information to the controller, the controller configured to adjust one or more of rate, waveform, or depth of the at least two cycles of negative pressure ventilation based at least in part on the information received from the user interface.

10. The medical device of claim 1, wherein the controller is further configured to perform high frequency ventilation, the high frequency ventilation including performing a ventilation cycle at frequency greater than or equal to three hertz, each ventilation cycle including:
positioning the piston at a reference position;
extending the piston from the reference position to a compression position to compress a chest of a patient; and
returning the piston from the compression position to the reference position.

11. The medical device of claim 10, wherein the frequency is between three hertz and fifteen hertz.

12. The medical device of claim 10, wherein the piston extended from reference position to the compression position is less than or equal to 1.5 inches.

13. The medical device of claim 12, wherein the piston extended from reference position to the compression position is more than 0.5 inches but less than 1.5 inches.

14. The medical device of claim 1, wherein the controller is further configured to perform high frequency ventilation, the high frequency ventilation including performing a ventilation cycle at frequency greater than or equal to three hertz, each ventilation cycle including:
positioning the piston at a reference position;
retracting the piston from the reference position to an expansion position to compress a chest of a patient; and
returning the piston from the expansion position to the reference position.

15. The medical device of claim 14, wherein the frequency is between three hertz and fifteen hertz.

16. The medical device of claim 14, wherein the piston extended from reference position to the compression position is less than or equal to four centimeters.

17. The medical device of claim 16, wherein the piston extended from reference position to the compression position is more than one centimeter but less than four centimeters.

18. A medical device, comprising:
a negative pressure ventilation mechanism configured to perform successive negative pressure ventilations on a chest of a patient, the negative pressure ventilation mechanism including a support portion configured to be placed underneath a patient, a piston, a patient contact member attached to the piston and configured to adhere to the patient's body, and a driver coupled to the piston configured to retract and extend the piston; and
a controller communicatively coupled with the negative pressure ventilation mechanism, the controller configured to:
receive at least one input;
determine whether at least one of a depth, a waveform, and a rate of negative pressure ventilation should be adjusted based on the at least one input;
responsive to a determination that at least one of the depth, the waveform, or the rate of negative pressure ventilation should be adjusted, cause the driver to adjust at least one of the depth, the waveform, or the rate of negative pressure ventilation.

19. The medical device of claim 18, further comprising:
a user interface configured to transmit the at least one input to the controller.

20. The medical device of claim 18, further comprising:
a sensor configured to transmit the at least one input to the controller.

21. The medical device of claim 18, wherein the at least one input includes blood oxygen saturation (SpO2) of the patient.

22. The medical device of claim 18, wherein the at least one input includes End-Tidal CO2 (ETCO2) of the patient.
